(19) United States
(12) Patent Application Publication
(10) Pub. No.: US 2007/0141123 A1
(43) Pub. Date: Jun. 21, 2007

(54) EMULSIONS COMPRISING NON-ESTERIFIED PHYTOSTEROLS IN THE AQUEOUS PHASE

(75) Inventor: Jerzy Zawistowski, Vancouver, BC (CA)

Correspondence Address: KIRTON AND MCCONKIE, 60 EAST SOUTH TEMPLE,, SUITE 1800, SALT LAKE CITY, UT, 84111, US

(73) Assignee: FORBES MEDI-TECH INC., Vancouver (CA)

(21) Appl. No.: 11/613,818

(22) Filed: Dec. 20, 2006

Related U.S. Application Data
(60) Provisional application No. 60/751,879, filed on Dec. 20, 2005.

(51) Int. Cl. A61K 47/00; A61K 31/56

(52) U.S. Cl. 424/439; 514/171

(57) ABSTRACT

A stable emulsion in which an aqueous phase has one or more phytosterols or phytostanols, or mixtures thereof in free (non-esterified) form and an oil or fat phase has phytosterols or phytostanols, or mixtures thereof, in esterified form.

### CROSS-REFERENCE TO RELATED APPLICATION

This application claims the benefit of U.S. Provisional Patent Application 60/751,879, filed Dec. 20, 2005.

### FIELD OF THE INVENTION

This present invention relates to compositions useful in the field of lipid disorders.

### BACKGROUND OF THE INVENTION

While recent advances in science and technology are helping to improve quality and add years to human life, the prevention of atherosclerosis, the underlying cause of cardiovascular disease (“CVD”) has not been sufficiently addressed. In fact, cardiovascular diseases account for more deaths annually than any other disease, including all forms of cancer combined¹. In the USA alone, more than one million heart attacks occur each year and more than half a million people die as a result. This enormous toll has necessitated continued research to determine the causes of CVD and means by which it can be prevented and treated.

The primary cause of CVD is atherosclerosis, a disease characterized by the deposition of lipids, including cholesterol, in the arterial vessel wall resulting in a narrowing of the vessel passages and ultimately a hardening of the vascular system. Atherosclerosis is a degenerative process resulting from an interplay of inherited (genetic) factors and environmental factors such as diet and lifestyle. Research to date suggest that cholesterol may play a role in atherosclerosis by forming atherosclerotic plaques in blood vessels, ultimately cutting off blood supply to the heart muscle or alternatively to the brain or limbs, depending on the location of the plaque in the arterial tree^{1,2}. A total cholesterol in excess of 225-250 mg/dl is associated with significantly elevated risk of CVD, including vascular disease. Overviews have indicated that a 1% reduction in a person's total serum cholesterol yields a 2% reduction in risk of a coronary artery event⁴. Statistically, a 10% decrease in average serum cholesterol (e.g. from 6.0 mmol/L to 5.3 mmol/L) may result in the prevention of 100,000 deaths in the United States annually⁵.

Cholesteryl esters are a major component of atherosclerotic lesions and the major storage form of cholesterol in arterial wall cells. Formation of cholesteryl esters is also a step in the intestinal absorption of dietary cholesterol through homeostatic control mechanisms. These control mechanisms involve the inter-related regulation of dietary cholesterol, cholesterol biosynthesis and catabolism of cholesterol-containing plasma lipoproteins. Cholesterol biosynthesis and catabolism occur primarily in the liver and hence, it is a prime determinant of plasma cholesterol levels.

Lipoproteins are complexes of lipids and proteins held together by non-covalent bonds. Each type of lipoprotein class has a characteristic mass, chemical composition, density and physiological role. Irrespective of density or particle size, circulating lipids consist of a core of cholesteryl esters and triglycerides, and an envelope of phospholipids, free cholesterol and apolipoproteins. The apolipoproteins are involved in the assembly and secretion of the lipoprotein, provide structural integrity, activate lipoprotein-modifying enzymes, and are the ligand for a large assortment of receptors and membrane proteins. Lipoprotein classes found in plasma include HDL, LDL, intermediate density lipoproteins (IDL) and very low density lipoproteins (VLDL).

Each type of lipoprotein has a characteristic apolipoprotein composition or ratio. The most prominent apolipoprotein in HDL is apolipoprotein-AI (apo-AI), which accounts for approximately 70% of the protein mass, with apo-AII accounting for another 20%. The ratio of apoA-I to apoA-II may determine HDL functional and anti-atherogenic properties. Circulating HDL particles consist of a heterogeneous mixture of discoidal and spherical particles with a mass of 200 to 400 kilo-daltons and a diameter of 7 to 10 nm.

HDL is one of the major classes of lipoproteins that function in the transport of lipids in plasma, and has multiple functions within the body, including reverse cholesterol transport, providing the cholesterol molecule substrate for bile acid synthesis, transport of clusterin, transport of paraoxanase, prevention of lipoprotein oxidation and selective uptake of cholesterol by adrenal cells. The major lipids associated with HDL include cholesterol, cholesteryl ester, triglycerides, phospholipids and fatty acids. HDL is anti-atherogenic.

The atherosclerotic process begins when LDL becomes trapped within the vascular wall. Oxidation of this LDL results in the binding of monocytes to the endothelial cells lining the vessel wall. These monocytes are activated and migrate into the endothelial space where they are transformed into macrophages, leading to further oxidation of the LDL. The oxidized LDL is taken up through the scavenger receptor on the macrophage, leading to the formation of foam cells. A fibrous cap is generated through the proliferation and migration of arterial smooth muscle cells, thus creating an atherosclerotic plaque.

HDL is essential for the transport of cholesterol from extra-hepatic tissues to the liver, where it is excreted into bile as free cholesterol or as bile acids that are formed from cholesterol. The process requires several steps. The first is the formation of nascent or pre-beta HDL particles in the liver and intestine. Excess cholesterol moves across cell membranes into the nascent HDL through the action of the ABCA transporter. Lecithin cholesterol acyl transferase (LCAT) converts the cholesterol to cholesteryl ester and the subsequent conversion of nascent HDL to mature HDL. Esterified cholesterol is then transferred by cholesteryl ester transfer protein (CETP) from HDL to apolipoprotein-B containing lipoproteins, which are taken up by numerous receptors in the liver. Nascent HDL is regenerated via hepatic triglyceride lipase and phospholipid transfer protein and the cycle continues. In addition to the cholesterol removed from peripheral cells, HDL accepts cholesterol from LDL and erythrocyte membranes. Another mechanism of reverse cholesterol transport may involve passive diffusion of cholesterol between cholesterol-poor membranes and HDL or other acceptor molecules.

HDL protects against the development of atherosclerosis both through its role in reverse cholesterol transport and possibly by impeding LDL oxidation. Several HDL-associated enzymes are involved in the process. Paroxonase (PONI), LCAT, and platelet activating factor acetylhydrolase (PAFAH) all participate by hydrolyzing phospholipid hydroperoxides generated during LDL oxidation and act in tandem to prevent the accumulation of oxidized lipid in LDL. These enzymes are responsible for the anti-oxidative and anti-inflammatory properties of HDL. Studies have shown that a low plasma concentration of HDL cholesterol is a significant risk factor for the development of atherosclerosis and that high levels are protective.

The liver is the major organ responsible for synthesis and secretion of VLDLs, which, as noted above, are metabolized to LDL in circulation. LDLs are the predominant cholesterol carrying lipoproteins in plasma and hence an increase in their concentration is directly correlated with atherosclerosis. Simply put, when intestinal cholesterol absorption is reduced, by any means, less cholesterol is delivered to the liver. As a result, VLDL production is reduced and there is a concomitant increase in hepatic clearance of plasma cholesterol, mostly in the form of LDL.

Accordingly, cholesterol acts on three different levels to regulate its own synthesis. Firstly, it suppresses endogenous cholesterol synthesis by inhibiting the enzyme HMG CoA reductase. Secondly, it activates LCAT. Thirdly, it regulates the synthesis of the LDL-receptor ensuring that a cell already having a sufficient amount of cholesterol will not take up additional cholesterol.

Sterols are naturally occurring compounds that perform many critical cellular functions. Phytosterols such as campesterol, stigmasterol and beta-sitosterol in plants, ergosterol in fungi and cholesterol in animals are each primary components of cellular and sub-cellular membranes in their respective cell types. The dietary source of phytosterols in humans comes from plant materials i.e. vegetables and plant oils. The estimated daily phytosterol content in the conventional western-type diet is approximately 60-80 milligrams in contrast to a vegetarian diet which would provide about 500 milligrams per day.

Phytosterols have received a great deal of attention due to their ability to decrease serum cholesterol levels when fed to a number of mammalian species, including humans. While the precise mechanism of action remains largely unknown, the relationship between cholesterol and phytosterols is apparently due in part to the similarities between the respective chemical structures (the differences occurring in the side chains of the molecules). It is assumed that phytosterols displace cholesterol from the micellar phase and thereby reduce its absorption or possibly compete with receptor and/or carrier sites in the cholesterol absorption process.

Over forty years ago, Eli Lilly marketed a sterol preparation from tall oil and later from soybean oil called Cytellin™ which was found to lower serum cholesterol by about 9% according to one report⁶. Various subsequent researchers have explored the effects of sitosterol preparations on plasma lipid and lipoprotein concentrations⁷ and the effects of sitosterol and campesterol from soybean and tall oil sources on serum cholesterols⁸. A composition of phytosterols which has been found to be highly effective in lowering serum cholesterol is disclosed in U.S. Pat. No. 5,770,749 to Kutney et al. and comprises no more than 70% by weight beta-sitosterol, at least 10% by weight campesterol and stigmastanol (beta-sitostanol). It is noted in this patent that there is some form of synergy between the constituent phytosterols, affording even better cholesterol-lowering results than had been previously achieved.

Despite the obvious and now well recorded advantages of phytosterols, not only in the treatment of CVD and its underlying conditions such as hypercholesterolemia, hyperlipidemia, atherosclerosis, hypertension, thrombosis but in the treatment of other diseases such as Type II diabetes, dementia cancer and aging, the administration of phytosterols and the incorporation thereof into foods, pharmaceuticals and other delivery vehicles has been complicated by the fact that they are highly hydrophobic (i.e. they have poor water solubility). This highly hydrophobic nature of phytosterols renders them insoluble and barely dispersible in aqueous media. As such, phytosterols tend to be added to the fat phase of fat-based food products. Health-conscious consumers wishing to benefit from the cholesterol lowering effects of phytosterols are therefore forced to consume fat-rich foods, despite the health risks of a high fat diet.

Attempts have been made to solve these problems using, for example, chemical modification of the phytosterols. For example, as noted above, esterification of phytosterols generally results in lowered melting temperatures. Thus, such phytosterol esters generally may be incorporated into food products more readily due to the lower melting points and can provide food products without significantly gritty texture. Although the problem of fat solubility of phytosterols can be improved by esterification, this is not a completely satisfactory solution to the problem for two reasons: 1) phytosterol esters are biologically less effective than non-esterified phytosterols; and 2) when a phytosterol or phytosterol ester is distributed within the small volume lipid phase of a low fat emulsified product, the taste of the product is adversely affected, since the high concentration of phytosterol in the fat leads to a waxy sensation in the mouth and on the tongue.

Earlier researchers attempted to overcome formulation limitations in relation to phytosterols by using several methods including: homogenization, encapsulation, and/or the addition of stabilizers, gums and the like. However, these methods increase the cost of the product, and in some instances are illegal in certain standardized products such as citrus juices.

Vulfson et al., WO 00/41491 discloses hydrophobic compounds such as plant sterols and lycopenes as supplements to food products and beverages such as oleomargarine products, drinks, soups, sauces, dips, salad dressings, mayonnaise, confectionery products, breads, cakes, biscuits, breakfast cereals and yogurt type products. Vulson et al., in combining the plant sterol or lycopene with the food product, theorizes that the food product which has both hydroxyl and carboxyl groups interacts with the surface of the sterol or lycopene.

Haarasilta et al., WO 98/58554, describes a premix used in the food industry containing a pulverized plant sterol and a conventional foodstuff ingredient such as fruit, vegetable or berry type of material, particularly in a powder form and methods for preparing the premix.

Zawistowski, WO 00/45648, describes a method of preparing microparticles of plant sterols and plant stanols or mixtures of both by dispersing and suspending the plant sterols and plant stanols in a semi-fluid, fluid or viscous vehicle and exposing the vehicle so formed to impact forces. The method involves dispersing or otherwise suspending the plant sterol and/or plant stanol in a suitable semi-fluid, fluid or viscous vehicle followed by applying impact forces to the vehicle to produce microparticles. Zawistowski develops these impact forces by creating high-shear either with an air atomization nozzle, a pneumatic nozzle, a high-shear mixer, or colloid mill, but preferably a microfluidizer commercially available from Microfluidics Incorporation, Newton, Massachusetts. Zawistowski observed that the plant sterols and/or plant stanols prepared in this way have greater “olubility” not only in oil based delivery systems but also in other media and can be incorporated into beverages such as colas, juices or dietary supplement and/or milk replacement drinks.

In order to increase the solubility of plant sterol, some researchers have synthesized various derivatives of plant sterol. For example, sitosterol mixed in certain ratios with starch hydrolysate, silicon dioxide and polyoxylene sorbitan monostearate through homogenizing, deaeration, pasteurizing and evaporation steps to form a medicinal powder for oral application, as disclosed in U.S. Pat. No. 3,881,005.

U.S. Pat. No. 5,932,562 discloses an aqueous homogeneous micellar mix of a plant sterol, lecithin and lysolecithin which has been dried to a finely divided water soluble powder. Other water-soluble plant sterols can be found in U.S. Pat. Nos. 6,054,144 and 6,110,502. According to these patents, aqueous-dispersible plant sterol is produced by admixing oryzanol or plant sterol, a monofunctional surfactant and polyfunctional surfactant in water at fixed ratios, and drying the admixture. This production method is characterized by being free from homogenization and deaeration steps with adoption of polyoxylene sorbitan monopalmitate and sorbitan monopalmitate as a monofunctional surfactant and a polyfunctional surfactant, respectively.

U.S. Pat. No. 6,190,720 discloses a food ingredient that can be used as a cholesterol-lowering agent, teaching that the food ingredient can be prepared by combining one or more molten plant sterols with one or more fats and one or more emulsifiers to homogeneity and cooling the homogeneous mixture to about 60° C. under agitation to give a paste. This food ingredient can be applied to oil-based foods such as salad dressings, margarine, etc.

PCT WO 00/33669 teaches that plant sterols can be dissolved or mixed in a melt of a food emulsifier, admixed with protein-containing foods such as milk or yogurt, homogenized, and added to food products. The dispersion stability of the cholesterol reducing, edible products is maintained only in the presence of a protein-containing material.

It is an object of the present invention to obviate or mitigate the above and other disadvantages as described further herein.

### SUMMARY OF THE INVENTION

The present invention provides a stable emulsion in which:
a) an aqueous phase comprises one or more phytosterols or phytostanols, or mixtures thereof in free (non-esterified) form; and
b) an oil or fat phase comprises phytosterols or phytostanols, or mixtures thereof, in esterified form.

The present invention further provides low fat emulsions in which:
a) an aqueous phase comprises one or more phytosterols or phytostanols, or mixtures thereof in free (non-esterified) form; and
b) an oil or fat phase comprises phytosterols or phytostanols, or mixtures thereof, in esterified form, said oil or fat phase comprising a reduced amount of fat due to the texturizing character of the free phytosterols or phytostanols, or mixtures thereof in the aqueous phase.

The present invention further provides a method of reducing fat in a food based food comprising an emulsion which comprises providing in an aqueous phase of said emulsion a texturizing-sufficient amount of one or more phytosterols, phytostanols or mixtures thereof.

The present invention further provides a method of treating or preventing cardiovascular disease and its underlying conditions including atherosclerosis, hyperlipidemic conditions, dyslipidemia, hypoalphalipoproteinemia, hypertension, and hypercholesterolemia in an animal which comprises administering a therapeutically effective amount of the emulsion described herein.

The invention further provides foods, beverages, nutraceuticals and pharmaceuticals of which the unique emulsion of the present invention forms all or part.

### PREFERRED EMBODIMENTS OF THE INVENTION

The following detailed description is provided to aid those skilled in the art in practising the present invention. However, this detailed description should not be construed so as to unduly limit the scope of the present invention. Modifications and variations to the embodiments discussed herein may be made by those with ordinary skill in the art without departing from the spirit or scope of the present invention.

As used herein, “animal” means any member of the animal kingdom, including preferably humans.

As used herein, “food” means any safe, ingestible product for animal use, including human use, and includes “functional foods”, “designer foods” and “pharmafoods”.

As used herein, “functional food” means a product that is similar in appearance to conventional foods that is consumed as part of a usual diet, but which has demonstrated physiological benefits and/or reduces the risk of disease beyond basic nutritional functions.

As used herein, “nutraceutical” means a non-pharmaceutical product prepared in the form of pills, powders, potions and in other medicinal forms not generally associated with food but which has a physiological benefit or provides protection against disease.

Anywhere in the world, nutraceuticals, functional foods, designer foods, and pharmafoods are synonyms for food or food ingredients considered to provide medical or health benefits, including the prevention and treatment of disease.

According to the present invention, there is provided a composition for incorporation into foods, beverages, nutritional or vitamin supplements, and nutraceuticals, which comprises one or more phytosterols and/or phytostanols and glucomannan.

The elements of the composition will be described in more detail below.

Phytosterols/Phytostanols

As used herein, the term “phytosterol” includes all sterols without limitation, for example: sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers. The term “phytostanol” refers to saturated or hydrogenated sterols including all natural or synthesized forms and derivatives thereof, and isomers. It is to be understood that modifications to the phytosterols and phtostanols i.e. to include side chains also falls within the purview of this invention. For example, the purview of this invention clearly includes 24 beta-ethylsitostanol, 24-alpha-ethyl-22-dehydrositostanol. It is also to be understood that, when in doubt throughout the specification, and unless otherwise specified, the term “phytosterol” encompasses both sterol and stanol. In a most preferred form, the sterol is in its saturated form and is a sitostanol, preferably beta-sitostanol.

These sterols and stanols for use in accordance with this invention may be procured from a variety of natural sources. For example, they may be obtained from the processing of plant oils (including aquatic plants) such as corn oil and other vegetable oils, wheat germ oil, soy extract, rice extract, rice bran, rapeseed oil, sunflower oil, sesame oil and fish (and other marine-source) oils. They may also be derived from fungi, for example ergosterol. Accordingly, the present invention is not to be limited to any one source of sterols. U.S. Pat. No. 4,420,427 teaches the preparation of sterols from vegetable oil sludge using solvents such as methanol. Alternatively, phytosterols and phytostanols may be obtained from tall oil pitch or soap, by-products of forestry practises as described in U.S. Pat. No. 5,770,749, incorporated herein by reference.

Within the scope of the present invention the “ester” of phytosterols and phytostanols refers to the form in which the free phytosterol or phytostanol is esterified prior to formation of the emulsion described herein.

To form phytosterol and/or phytostanol esters, many methods are known in the art. For example, one or more suitable aliphatic acids or their esters with low boiling alcohols may be condensed with the selected phytosterol and/or phytostanol. A wide variety of aliphatic acids or their esters may be used successfully and include all aliphatic acids consisting of one or more alkyl chains with one or more terminal carboxyl groups. These aliphatic acids may be natural of synthetic and are represented by the following chemical formulae:
a) R1-COOH (monocarboxylic acid) wherein:
   R1 is an unbranched saturated alky group, represented by CH₃—, CH₃CH₂— or CH₃(CH₂)ₙCH₂— WHERE n=3-25; or
   R1 is a branched saturated alkyl group represented by CₙH₂ₙ₊₁- where n=1-25 is the number of carbon atoms contained in the group R1; the branching typically refers, but is not limited to one or more methyl group side chains (branches); or
   R1 is an unbranched or branched unsaturated alkyl group, represented by the formula CₙH₂ₙ₋₂ₘ₊₁, where n=1-25 is the number of carbon atoms in R1 and m=degree of unsaturation; or
b) HOOC—R2-COOH is a dicarboxylic acid wherein:
   R2 is an unbranched saturated alkly group, represented by —CH₂—, or -_{CH2CH2}-, or —CH₂(CH₂)ₙCH₂ where n=3-25; or
   R2 is a branched saturated alkyl group represented by —CₙH₂ₙ— where n=1-25 is the number of carbon atoms contained in the group R2; the branching typically refers, but is not limited to, one or more methyl group side chains (branches); or
   R2 is an unbranched or branched unsaturated alkyl group, represented by the formula CₙH₂ₙ₋₂ₘ, where n=1-25 is the number of carbon atoms in R2 and m=degree of unsaturation; or
c) a tricarboxylic acid represented by the formula: 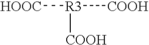
   wherein, in this formula:
   R3 is a branched saturated alkyl group represented by —CₙH₂ₙ₋₁— where n=1-25 is the number of carbon atoms contained in the group R3; the branching typically refers, but is not limited to, one or more methyl group side chains (branches); or
   R3 is a branched unsaturated alkyl group, represented by CₙH₂ₙ₋₂ₘ₋₁— wherein n=1-25 is the number of carbon atoms in R3 and m=the degree of unsaturation; or
d) a mono-. di-, or tricarboxylic acid as defined above, which may contain one, two or three hydroxyl groups in the molecule.
In a preferred form, the acid is either a straight-chain or branched unsaturated or saturated, aliphatic or aromatic acid. More preferably, the acids are selected, inter alia, from the following list: valeric acid, isovaleric acid, sorbic acid, isocaproic acid, lauric acid, myrestic acid, palmitic acid, stearic acid, caproic acid, ascorbic acid, arachidic acid, behenic acid, hexacosanoic acid, octacosanoic acid, pentadecanoic acid, erucic acid, linoleic acid, linolenic acid, arachidonic acid, acetic acid, citric acid, tartaric acid, palmitoleic acid and oleic acid. The most preferable fatty acids within the scope of the present invention are linoleic acid, linolenic acid and arachidonic acid which may be obtained from natural sources such as safflower oil, sunflower oil, olive oil and corn oil (linoleic acid), safflower oil, sunflower oil, olive oil and jojoba oil (linolenic acid and arachidonic acid) and rapeseed oil (erucic acid).

Other aromatic acids are clearly contemplated within the scope of the present invention.

To form a phytosterol ester in accordance with the present invention, the selected phytosterol and acid or its ester with volatile alcohol may be mixed together under reaction conditions to permit condensation of the phytosterol with the acid to produce an ester. A most preferred method of preparing these esters which is widely used in the edible fat and oil industry is described in U.S. Pat. No. 5,502,045 (which is incorporated herein by reference). As no substances other than the free phytosterol, a fatty acid ester or mixture thereof and an interesterification catalyst like sodium ethylate are used, the technique is highly suitable for preparing products ultimately for human consumption. In overview, this preferred method, adapted for use within the present invention, comprises heating the phytosterol(s) with a vegetable oil fatty acid ester (preferably a methyl ester) at a temperature from 90-120° C. and subsequently adding a suitable catalyst such as sodium ethylate. The catalyst is then removed/destroyed by any one of the techniques known in the art e.g. adding water and/or filtration/centrifugation.

Another method which may be used in accordance with the present invention is described in U.S. Pat. No. 4,588,717, which is also incorporated herein by reference. A preferred method is to mix the phytosterol and the fatty acid together bringing the mixture to a temperature of from about 15° C. to about 45° C. at about atmospheric pressure for approximately one to three hours.

Accordingly, it is to be understood that the widest possible definition is to be accorded to the terms “phytosterol ester” and “phytostanol ester” as used herein, including, but not limited to: esterified phytosterols and phytostanols with aliphatic or aromatic acids (thereby forming aliphatic or aromatic esters, respectively), phenolic acid esters, cinnamate esters, ferulate esters, phytosterol and phytostanol glycosides and acylated glycosides or acylglycosides.

It has been found that emulsions of the present invention overcome various industry problems related to “healthful” fat-based or emulsion-based food products. It is considered desirable to include free phytosterols and phytostanols in foods and other consumer deliverables, as opposed to their esterifed counterparts, due to the fact that:
1) phytosterol esters are biologically less effective than non-esterified phytosterols; 2) when a phytosterol or phytosterol ester is distributed within the small volume lipid phase of a low fat emulsified product, the taste of the product is adversely affected, since the high concentration of phytosterol in the fat leads to a waxy sensation in the mouth and on the tongue; and 3) additional fat is provided in to product in order to prepare and deliver the fatty acid esterifed form of phytosterols/phytostanols.

Despite these issues, conventionally, as discussed above, phytosterols and phytostanols in their esterified form are added to fat based foods and to the fat or oil phase of emulsions, often in preference and to the almost complete exclusion of phytosterols and phytostanols in their free form. Furthermore, when phytosterols or phytostanols are added, they are mixed with the phytosterol or phytostanol esters in the fat moiety or oil phase. The present invention teaches that this does not need to be the case.

Examples of conventional practice with respect to emulsions such as yellow fat spreads include:

EP 0898896 (Unilever), which describes a fat based food product comprising natural fat components which have a blood cholesterol lowering effect wherein the fat comprises at least 1% of a sterol composition which comprises sterols of which at least 40% is esterified with fatty acid esters. These sterols were esterified with fatty acids from sunflower seed oil with an esterification rate of 65%. The sterol-ester concentrates were used in spread production together with other edible oils and fats. Final sterol equivalent concentration (as free and esterified sterol) was 11.0% on product.

Fat blends used in the production of fat-containing products like margarines, spreads and spreadable cheeses, consist of a liquid oil fraction and a so-called hardstock. The liquid oil fraction typically comprises liquid unmodified vegetable oils such as soybean oil, sunflower oil, low erucic acid rapeseed oil (Canola), corn oil and blends of vegetable oils. Hardstock typically comprises a blend of fats that are solid at room temperature. The hardstock contais a high proportion of triglycerides that crystallize to give the final product certain desired physical properties such as texture, creaminess and melt-down in the mouth. Texture typically encompasses a number of desired characteristics such as viscosity, plasticity, solid fat content versus temperature and melting point.

A number of efforts have been undertaken in an attempt to replace at least a portion of the hardstock with other ingredients that are capable of contributing the same sensory benefits to the food product without the undesirable side effects of the saturated fatty acids and trans fatty acids. U.S. Pat. No. 5,354,573 teaches the use of fat-soluble polymers as textunizers in foods.

Another approach to obtaining a healthier fatty acid profile of the fat blend to be used in fat-containing products is to alter the composition of the hardstock to reduce to a minimum the levels of fatty acids such as launic acid and mynistic acid. Fatty acids of this type are known for their potential for increasing cholesterol levels in the blood. Typically, the hardstock is produced by co-interesterification of a fully hydrogenated vegetable oil with liquid unsaturated vegetable oils. This procedure is discussed in the Journal of the American Oil Chemists' Society (AOCS) 72, (1995), page 379-382.

Others have attempted to reduce the fat-content of margarines or spreads by the use of stabilizers such as gelatin, pectin, oligofructose and different gels such as xanthan gum, guar gum, alginate, carrageenan and cellulose derivatives. Other fat replacers have also been used in an attempt to mimic the mouth feel of the final product while reducing its total content of saturated and trans-unsaturated fat. U.S. Pat. No. 5,502,045 discloses the use of sitostanol fatty acid esters for reducing the absorption of cholesterol. Example 5 of the patent describes a margarine which contains 80% of a fat composed of 60% rapeseed oil, 35% partially hardened soybean oil and 5% coconut oil. β-sitostanol fatty acid ester in an amount of 10% and 20% by weight of the fat was added as a diluent to the fat blend diluting both the liquid part of the fat blend as well as the hardstock.

In WO 9819556 (Raisio), it was found that stanol and sterol fatty acid esters or their blends, defined as texturizing agents, form crystal networks with sirmilar properties as those of conventional hardstock triglycerides. They suggested that these texturizing agents could be used fully or partly as replacements for the conventional hardstock in fat blends to be used in fat containing products. There was no mention of the use of free phytosterols or phytostanols in this role. The significant disadvantage to this technology is that, while less hardstock may be required as esters are used in replacement, the fact remains that the esters have a fat component themselves. In essence, it replaces one fat with another.

It has been surprisingly found, within the scope of the present invention, that free, non-esterifed phytosterols, phytostanols or mixtures of both, when incorporated into the aqueous phase of an emulsion, provide significant texturizing properties (i.e. meaning that reduced amounts of hardstock or fat may then be required) and reduce the amount of phytosterol ester or phytostanol ester in the fat or oil phase. By this means, actual total fat in the emulsion may be reduced.

In a preferred embodiment, in fat based foods which are emulsions, the free phytosterol/stanol moiety is added to the aqueous phase of the emulsion, during manufacture, in an amount by weight which is greater than the amount by weight of the phytosterol/stanol esters added to the fat or oil phase of the emulsion during manufacture.

In a further preferred embodiment, in fat based foods which are emulsions, the free phytosterol/stanol moiety is added to the aqueous phase of the emulsion, during manufacture, in an amount by weight which is from 5-95% greater than the amount by weight of the phytosterol/stanol esters added to the fat or oil phase of the emulsion during manufacture.

Formation of Emulsions:

Emulsions are finely divided or colloidal dispersions comprising two immiscible phases, e.g. oil and water, one of which (the internal or discontinuous phase) is dispersed as droplets within the other (external or discontinuous phase). Thus an oil-in-water emulsion consists of oil as the internal phase, dispersed water as the external phase, the water-in-oil emulsion being the opposite.

A wide variety of emulsified systems may be formed in accordance with the present invention (i.e. having free phytosterols/stanols in the aqueous phase and esterified phytosterols/stanols in the fat phase) including standard emulsions of all types and microemulsions.

Generally, emulsions may include oil and water phases, emulsifiers, emulsion stabilizers and optionally preservatives, flavouring agents, pH adjusters and buffers, chelating agents, antifoam agents, tonicity adjusters and anti-oxidants. Suitable emulsifiers (wherein bracketed numerals refer to the preferred HLB values) include: anionic surfactants such as alcohol ether sulfates, alkyl sulfates (30-40), soaps (12-20) and sulfosuccinates; cationic surfactants such as quaternary ammonium compounds; zwitterionic surfactants such as alkyl betaine derivatives; amphoteric surfactants such as fatty amine sulfates, difatty alkyl triethanolamine derivatives (16-17); and nonionic surfactants such as the polyglycol ether derivatives of aliphatic or cycloaliphatic alcohols, saturated fatty acids and alkyphenols, water-soluble polyethyleneoxy adducts onto polypropylene glycol and alkyl polypropylene glycol, nonylphenol polyethoxyethanols, castor oil polyglycol ethers, polypropylene/polyethylene oxide adducts, tributylphenoxy-polyethoxyethanol, polyethylene glycol, octylphenoxypolyethoxyethanol, lanolin alcohols, polyoxyethylated (POE) alkyl phenols, POE fatty amides, POE fatty alcohol ethers, POE fatty amines, POE fatty esters, poloxamers (719), POE glycol monoethers (13-16), polysorbates and sorbitan esters. This list is not intended to be exhaustive as other emulsifiers are equally suitable.

Appropriate emulsion stabilizers include, but are not limited to, lyophilic colloids such as polysaccharides (e.g. acacia, agar, alginic acid, carrageenin, guar gum, karaya gum, tragacanth xanthan gum), amphoterics (e.g. gelatin) and synthetic or semi-synthetic polymers (e.g. carbomer resins, cellulose ethers, carboxymethyl chitin, polyethylene glycol-n (ethylene oxide polymer H(OCH2CH2)nOH); finely divided solids including clays (e.g. attapulgite, bentonite, hectorite, kaolin, magnesium aluminum silicate and montmorillonite), microcrystalline cellulose oxides and hydroxides (e.g. aluminum hydroxide. magnesium hydroxide and silica); and cybotactic promoters/gellants including amino acids, peptides, proteins lecithin and other phospholipids and poloxamers. Suitable anti-oxidants for use in the formation of emulsions include: chelating agents such as citric acid, EDTA, phenylalanine, phosphoric acid, tartaric acid and tryptophane; preferentially oxidized compounds such as ascorbic acid, sodium bisuffite and sod ium sulfite; water soluble chain terminators such as thiols and lipid soluble chain terminators such as alkly gallates, ascorbyl paimitate, t-butyl hydroquinone, butylated hydroxyanisole, butylated hydroxytoluene, hydroquinone, nordihydroguaiaretic acid and alpha-tocopherol. Suitable preservatives, pH adjustment agents, and buffers, chelating agents, osmotic agents, colours and flavouring agents may be added.

In a most preferred form, an emulsifier with a high HLB value is selected for use in the aqueous phase. The HLB system is a scale used for describing the characteristics of a surface-active agent. Detailed information on the HLB system and determination of HLB values can be found in the Kirk-Othmer Encyclopedia of Chemical Technology, (3 Ed.) 8: pp 910-918, which is incorporated herein by reference. Emulsifiers having HLB values in the range 7 to 18, especially 8 to 18, are often termed oil in water (o/w) emulsifiers. W/o emulsifiers have HLB values in the range 1-9, especially 1-6. Since HLB numbers are additive, the overall HLB value of a blend of emulsifiers of known HLB can easily be calculated.

In one preferred form of the present invention, any non-sterol emulsifier used in the aqueous phase has an HLB value equal or greater than 17.

In general, such emulsifers include alkoxylate emulsifiers with an average of from about 10 to about 100 alkylene oxide, particularly ethylene oxide residues; and non-alkoxylate emulsifiers including sugar mono-esters and poiyglycerol mono-esters, hydrocarbyl, especially alkyl, polysaccharides; fatty acid glycerol esters where the fatty acid has 8 to 12 carbon atoms such as glycerol mono-laurate and fatty acid N-sugar amides such as glucamides. However, any type of liquid emulsifier meeting the HLB requirement can be used. Examples of other emulsifiers of this type can be found in McCutcheon's, Vol 1: Emulsifiers & Detergents, 2000, the contents of which are incorporated herein by reference.

Specific examples of emulsifiers which may be selected for use in the aqueous phase, in accordance with one preferred aspect of the present invention include, but are not limited to: sodium stearoyl lactylate (“SSL” HLB 21), sucrose monostearate, HLB 16; sucrose monolaurate, sodium oleate HLB 18, calcium stearoyl lactylate; sodium oleate (HLB 18); polyoxyethylene-20-sorbitan monopalmitate (HLB 15.6); polyoxyethylene-40-stearate (HLB 16.9); Tween 20 (POE (20) sorbitan monolaurate) (HLB of about 16.7), polyoxyethylene sorbitan monopalmitate, and polyoxyethylene stearic acid monoester. Additionally, Emultop™ may be used.

The most preferred non-sterol emulsifier for use within the scope of the present invention is SSL, which can be made by combining lactic acid and stearic acid, and then reacting the result with sodium hydroxide or calcium hydroxide to make the sodium or calcium salt

The general preparation of emulsions is as follows: the two phases (oil and water) are separately heated to an appropriate temperature (the same in both cases, generally 5′I OoC above the melting point of the highest melting ingredients in the case of a solid or semi-solid oil, or where the oil phase is liquid, a suitable temperature as determined by routine experimentation). Water-soluble components are dissolved in the aqueous (water) phase and oil-soluble components are dissolved in the oil phase. To create an oil-in water emulsion, the oil phase is vigorously mixed into the aqueous phase to create a suitable dispersion and the product is allowed to cool at a controlled rate with stirring.

A water-in-oil emulsion is formed in the opposite fashion i.e. the water phase is added to the oil phase. When hydrophillic colloids are a part of the system as emulsion stabilizers, a phase inversion technique may be employed whereby the colloid is mixed into the oil phase rather than the aqueous phase, prior to addition to the aqueous phase. In using the oil-based composition of the present invention, which is semi-solid, it is preferred to add the composition to the oil phase prior to heating.

Microemulsions, characterized by a particle size at least an order of magnitude smaller (10-100 nm) than standard emulsions and defined as “a system of water, oil and amphiphile which is a single optically isotropic and thermodynamically stable liquid” (14), may also be formed comprising the composition of the present invention. In a preferred form, the microemulsion comprises a surfactant or surfactant mixture, a cosurfactant, (usually a short chain alcohol) the oil-based composition of the present invention, water and optionally other additives.

Surfactant or surfactant mixtures which are suitable for use in the formation of microemulsions can be anionic, cationic, amphoteric or non-ionic and possess HLB (hydrophile-lipophile balance) values within the range of 1-20, more preferably in the ranges 2-6 and 8-17. Especially preferred agents are non-ionic surfactants, selected from the group consisting of polyglycol ether derivatives of aliphatic or cycloaliphatic alcohols, saturated fatty acids and alkyphenols, water-soluble polyethyleneoxy adducts onto polypropylene glycol and alky polypropylene glycol, nonylphenol polyethoxyethanols, castor oil polyglycol ethers, polypropylene/polyethylene oxide adducts, tributylphenoxy-polyethoxyethanol, polyethylene glycol, octylphenoxypolyethoxyethanol, lanolin alcohols, polyoxyethylated (POE) alkyl phenols, POE fatty amides, POE fatty alcohol ethers, POE fatty amines, POE fatty esters, poloxamers (719), POE glycol monoethers (13-16), polysorbates and sorbitan esters.

There are many methods known and used by those skilled in the art for making microemulsions. In a preferred method of forming microemulsions of the present invention, a surfactant, a co-surfactant and the phytosterol, phytostanols or mixtures thereof (pre-dissolved in a suitable proportion of an appropriate oil) is mixed and then titrated with water until a system of desired transparency is obtained.

It is to be understood that, with the scope of the present invention, a broad definition should be accorded to the term “emulsion”. Generally, emulsions are defined as mixtures of at least two immiscible liquids—which in foods usually means oil and water. At least one of these is the continuous (or external) phase, within which you would find the dispersed (discontinuous, internal) phase. But the food industry also includes non-liquids in the definition—products such as breads, meat emulsions and ice cream. Meat emulsions are actually a gelled-protein oil-in-water (O/W) matrix of ground meat, emulsifiers and stabilizers. Bakery products generally are (excluding some fillings and cremes) O/W matrices of starch and protein networks supporting air bubbles. Butter and margarine are products built on water-in-oil (W/O) fat-crystal networks.

Important oil-in-water food emulsions, ones in which oil or fat is the dispersed phase and water is the continuous phase, include milk, cream, ice cream, salad dressings, cake batters, flavour emulsions, meat emulsions, and cream liquers. Examples of food water-in-oil emulsions are butter or margarine.

The emulsified fat based products created in accordance with the invention may be oil-in-water (o/w, or water continuous) or water-in-oil (w/o, or oil continuous) emulsions according to which specific emulsifiers may be are selected for use in preparing the emulsion. Water in oil (w/o or oil continuous emulsions), such as those made in production of spreads, margarines, icings, frostings etc. . . . benefit significantly from the incorporation of free phytosterols/stanols into the aqueous phase not only for the enhanced therapeutic efficacies as compared to their corresponding esters, but also due to the unexpected but highly beneficial texturizing effect, which allows for a reduction in the amount of solid fat or hardstock required.

Methods of Use

The present invention provides a method of treating or preventing cardiovascular disease and its underlying conditions including atherosclerosis, hyperlipidemic conditions, dyslipidemia, hypoalphalipoproteinemia, hypertension, hypercholesterolemia and visceral obesity in an animal which comprises administering a therapeutically effective amount of an emulsion described herein.

The invention further provides a method of achieving at least one of the following prophylactic and treatment goals in an animal: lowering serum LDL cholesterol, increasing serum HDL cholesterol, and decreasing serum triglycerides, which comprises administering to the animal a therapeutically effective amount of an emulsion as described herein.

This invention further comprises the use of the disclosed emulsions for these indications, particularly, but not exclusively in the as or forming part of foods, beverages, nutraceuticals, and pharmaceuticals.

In particular, the emulsions of the present invention have been found to be especially useful in addressing at least two significant factors contributing to the multi-factorial presentation of cardiovascular disease: elevated LDL levels and elevated total cholesterol cholesterol levels.

The desired effects described herein may be achieved in a number of different ways. As noted above, the emulsions of the present invention may be administered by forming all or part of foods, beverages, nutraceuticals, foods including functional foods, and the like.

The term “therapeutically effective” is intended to qualify the combined amount of the phytosterols and/or phytostanols (in free and esterified form) taken by or administered to the animal, in particular the human, in order to achieve one or more of the following prophylactic and/or treatment goals:
a) lowering serum LDL cholesterol;
b) increasing serum HDL cholesterol;
c) decreasing serum triglycerides levels;
d) treating conditions associated with CVD generally;
e) treating atherosclerosis;
f) treating hypercholesterolemia;
g) treating a hyperlipidemic condition;
h) preventing, reducing, eliminating or ameliorating a dyslipidemic condition or disorder;
i) preventing, reducing, eliminating or ameliorating hypercholesterolemia, hypoalphalipoproteinemia,
j) preventing, reducing, eliminating or ameliorating the development of atherosclerotic lesions; and
k) preventing, reducing, eliminating or ameliorating any condition, disease or disorder which has as its basis or which is exacerbated by a deficiency in plasma HDL, or excess of either LDL, VLDL, Lp(a), beta-VLDL, IDL or remnant lipoproteins.

The emulsions of the present invention may be manufactured in a manner that is itself known, e.g., by means of conventional emulsion formation, with the exception thatmixing, dissolving, granulating, emulsifying, encapsulating, entrapping or lyophilizing processes.

In another form of the present invention, the emulsions of the present invention may form all or part of a foods, beverages, nutritional supplements and nutraceuticals, including, without limitation, the following:
1) Dairy Products—such as cheeses, butter, milk and other dairy beverages, spreads and dairy mixes, ice cream and yoghurt;
2) Fat-Based Products—such as margarines, spreads, mayonnaise, shortenings, cooking and frying oils and dressings;
3) Cereal-Based Products—comprising grains (for example, bread and pastas) whether these goods are cooked, baked or otherwise processed;
4) Confectioneries—such as chocolate, candies, chewing gum, desserts, non-dairy toppings (for example Cool Whip™), sorbets, icings and other fillings;
5) Beverages—whether alcoholic or non-alcoholic and including colas and other soft drinks, juice drinks, dietary supplement and meal replacement drinks such as those sold under the trade-marks Boost™ and Ensure™; and
6) Miscellaneous Products—including eggs and egg products, processed foods such as soups, pre-prepared pasta sauces, pre-formed meals and the like.

### EXAMPLE

The following example is provided by way of illustration of one aspect of the invention, and is not intended to limit the scope of the invention disclosed in its entirety.

### Example 1

### Spread

Light margarine (60% fat) containing 6% of Reducol was produced in batches of 5-10 kg. Reducol comprised campesterol, campestanol, β-sitosterol and sitostanol was added to the water (aqueous) phases. Clear fat solution (into which ester mixture comprising campesterol ester, campestanol ester, β-sitosterol ester and sitostanol ester was dissolved) was placed in the feeding tank (20 L), cooled to 40-45° C. and stirred using (Ultra-Turrax T50 equipped with the dispersing element S50N, IKA Works Inc., Wilmington, N.C., USA). Next, the water fraction (40%) was added and temperature was adjusted to 60° C. The blend was submitted into a votator and processed at 8-10° C. The composition of margarine is described below.

Reducol Spread, VK 56

| | | |
|---|---|---|
| | Amount per | |
| Ingredient | 1000 kg | % |

Fat Phase

| | | |
|---|---|---|
| rape seed oil | 288.803 | 28.88 |
| coconut oil | 35.000 | 3.50 |
| Magfat CAF 50 | 50.000 | 5.00 |
| Wood Sterol Ester | 37.200 | 3.72 |
| Mono-Di Glyceride | 3.000 | 0.30 |
| Lecithin | 0.800 | 0.08 |
| Beta-Carotin solution (˜1%) | 3.200 | 0.32 |
| Vitamin A/D3 premix | 0.042 | 0.00 |
| Vitamin D3 (1Mio IU) | 0.005 | 0.00 |
| Flavour A | 0.100 | 0.01 |
| Flavour B | 0.050 | 0.01 |
| Sum | 418.200 | 41.82 |

Water Phase

| | | |
|---|---|---|
| water | 482.700 | 48.27 |
| Reducol | 37.100 | 3.71 |
| starch | 42.000 | 4.20 |
| salt | 10.000 | 1.00 |
| lactic acid (80%) | 1.000 | 0.10 |
| citric acid | 0.200 | 0.02 |
| potassium sorbate | 1.000 | 0.10 |
| flavour | 0.800 | 0.08 |
| skim milk powder | 7.000 | 0.70 |
| Sum | 581.800 | 58.18 |

### REFERENCES:

1. Levi R I Declining Mortality in Coronary Hear Diseases Atherosclerosis 1981 1 312-325
2. Law M. R., Wald N. J., Wu., Hacksaw Z A., Bailey A.; Systemic underestimation of association between serum cholesterol concentration and ischemic heart disease in observational studies: Data from BUPA Study; Br. Med. J. 1994; 308:363-366
3. Law M. R., Wald N. J., Thompson S. G.; By how much and how quickly does reduction in serum cholesterol concentration lower risk of ischemic heart disease? Br. Med. J. 1994; 308:367-373
4. La Rosa J. C., Hunninghake D.. Bush D. et al.; The cholesterol facts: A summary of the evidence relating to dietary fats, serum cholesterol and coronary heart disease: A joint statement by the American Heart Association and the National Heart, Lung and Blood Institute. Circulation 1990; 81:1721-1733
5. Havel R. J., Rapaport E.. Drug Therapy: Management of Primary Hyperlipidemia. New England Journal of Medicine, 1995; 332:1491-1498
6. Lees A. M., Mok H. Y. I., Lees R. S., McCluskey M. A., Grundy S. M. Plant sterols as cholesterol-lowering agents: clinical trials in patients with hypercholesterolemia and studies of sterol balance. Atherosclerosis 1977; 28: 325-338
7. New Eng. J. Med., 336, 973-9 (1999)
8. J. Path. 181, 93-9 (1997)

1. A stable emulsion in which:
a) an aqueous phase comprises one or more phytosterols or phytostanols, or mixtures thereof in free (non-esterified) form; and
b) an oil or fat phase comprising phytosterols or phytostanols, or mixtures thereof, in esterified form.

2. The emulsion of claim 1 wherein the phytosterol is selected from the group consisting of sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol and all natural or synthesized forms and derivatives thereof, including isomers.

3. The emulsion of claim 1 wherein the phytostanol is selected from the group consisting of all saturated or hydrogenated phytosterols and all natural or synthesized forms and derivatives thereof, including isomers.

4. The emulsion of claim 1 wherein the phytosterols and/or phytostanols in the oil or fat phase are in form selected from the group consisting of: aliphatic acid esters, aromatic acid esters, phenolic acid esters, cinnamate esters, ferulate esters, phytosterol/phytostanol glycosides, and phytosterol/phytostanol acylglycosides.

5. The emulsion of claim 1 which is a yellow spread (margarine), butter, dressing, mayonnaise, shortening, filling, topping, cream, soup, whitener, or sauce.

6. A method for treating or preventing cardiovascular disease and its underlying conditions including atherosclerosis, hyperlipidemic conditions, dyslipidemia, hypoalphalipoproteinemia, hypertension, hypercholesterolemia and visceral obesity in an animal which comprises administering a therapeutically effective amount of the emulsion of claim 1.

7. The method of claim 6 wherein the animal is human.

8. A method of achieving at least one of the following therapeutic goals in an animal: lowering serum LDL cholesterol, increasing serum HDL cholesterol and decreasing serum triglycerides which comprises administering to the animal a therapeutically effective amount of the emulsion of claim 1.

9. A food having incorporated therein the emulsion of claim 1.

10. A beverage having incorporated therein the emulsion of claim 1.

11. A nutraceutical or functional food having incorporated therein the emulsion of claim 1.
